(19) United States
(12) Patent Application Publication
(10) Pub. No.: US 2025/0387616 A1
(43) Pub. Date: Dec. 25, 2025

(54) SKIN-ATTACHABLE TRANSPARENT ELECTRODE AND METHOD FOR MANUFACTURING SAME

(71) Applicant: Asen company, Seoul (KR)

(72) Inventors: Seung-Rok KIM, Seoul (KR); Ji-Wan JEON, Seoul (KR); Jin-Woo PARK, Seoul (KR)

(21) Appl. No.: 18/878,891

(22) PCT Filed: Aug. 23, 2023
(86) PCT No.: PCT/KR2023/012514

§ 371 Date: Dec. 25, 2024

(30) Foreign Application Priority Data
Aug. 25, 2022 (KR) 10-2022-0106789

(51) Int. Cl. A61N 1/04; C09D 183/08

(52) U.S. Cl. CPC A61N 1/0496; A61N 1/0468; C09D 183/08

(57) ABSTRACT

The present invention relates to a skin-attachable transparent electrode, a method for manufacturing the same, a wearable electronic device including the same, and a wound healing pad including the same. According to an aspect of the present invention, there is provided a skin-attachable transparent electrode including an alcohol-soluble biocompatible elastomer matrix and a conductive material network embedded in the matrix, in which one surface of the biocompatible elastomer matrix is dissolved upon contact with alcohol and is conformally coated on the skin.

### TECHNICAL FIELD

The present invention relates to a skin-attachable transparent electrode and a method for manufacturing the same.

### BACKGROUND ART

Recently, interest in and research on wearable electronic devices are increasing due to the development of the Internet of Things (IoT) and increased interest in well-being.

Note that a wound refers to a condition where the skin and subcutaneous tissue are damaged due to external stimuli. Most wounds are mild and can heal naturally, but there are chronic wounds that do not heal for 4 to 8 weeks and develop into ulcers extending beyond the depth of the skin and subcutaneous tissue. Patients with chronic diseases who have blood circulation disorders are particularly vulnerable to chronic wounds, making them prone to bacterial exposure even in small wounds, which makes it difficult to avoid secondary infections such as sepsis. Additionally, the dressing must be continuously changed to treat such wounds, and if the condition worsens, multiple surgical operations such as tissue removal and skin grafting are required. That is, chronic wounds not only increase the suffering of patients and their guardians, but also increase the financial burden of treatment, making their treatment increasingly important.

As research on treating chronic wounds continues to progress, approaches focusing on the natural wound healing mechanisms have been proposed to apply energy such as pressure, electrical stimulation, light, and ultrasound to chronic wound sites where natural healing is no longer possible, thereby enhancing cell migration and proliferation. Among these approaches, the effectiveness of a wound healing method that applies electrical stimulation to the wound site has been proven through continuous cell and animal experiments. Regarding this, hydrogel has been commercialized as an electrode material for direct attachment to the skin. However, hydrogel materials have low electrical conductivity and low transparency, which makes patients feel uneasy from an aesthetic perspective, and have a problem of reduced adhesive force caused by moisture evaporation.

Since electrode materials used in wearable electronic devices or wound treatment must adhere well to the body, the development of electrode materials with adhesiveness is very important. In particular, for biosensors that are attached to the body to detect body movements or biological signals, it is necessary not only to ensure firm attachment but also to achieve conformal contact with the skin. Therefore, electrode materials with excellent conformal contact properties with the skin are required. Skin-attachable electrode materials are required to have not only conformal contact properties with the skin but also transparency for biocompatibility and aesthetic use.

To achieve these properties, approaches have been proposed to utilize commercial adhesives or to impart adhesiveness to a surface of a skin-contact electrode while reducing an overall thickness of a device.

However, the approach of attaching an electrode to the skin using a commercial adhesive is unsuitable because it carries a high risk of causing skin irritation depending on the type of adhesive, and furthermore, the adhesive may obstruct direct contact between the electrode and the skin, thereby interfering with detection of electrical signals.

On the other hand, research is being conducted on thermoplastic polymers that dissolve in solvents, as the approach of imparting adhesiveness to the electrode material itself. For example, Non-Patent Literature 1 discloses an approach of providing adhesiveness and electrical properties by positioning silver nano-mesh and polyvinyl alcohol (PVA) on the skin and then dispersing water to utilize the water-soluble property of PVA. However, the above-described approach has a disadvantage in that the polymer is dissolved by sweat, which is mainly composed of water, thereby damaging the electrode or the device including the same. In addition, the approach has limitations in that sufficient conformal contact properties with the skin are not achieved due to a lifting phenomenon occurring between the attached electrode and the skin caused by movements such as bending the skin. Therefore, there is a need for a skin-attachable electrode material that not only has improved adhesiveness, biocompatibility, and transparency but also provides enhanced conformal contact properties with the skin without being affected by external environments such as sweat.

### CITATION LIST

(Non-Patent Literature 1) W.-H. Yeo, Y.-S. Kim, J. Lee, A. Ameen, L. Shi, M. Li, S. Wang, R. Ma, S. H. Jin, Z. Kang, Y. Huang, J. A. Rogers, Adv. Mater. 2013, 25, 2773

### DISCLOSURE

### Technical Problem

An object of the present invention is to provide a skin-attachable transparent electrode that has remarkably excellent adhesion to the skin, biocompatibility, light transmittance, and conformal contact properties with the skin, and at the same time, provides significantly improved stability because it does not dissolve by external environments such as sweat, and a method for manufacturing the same.

Another object of the present invention is to provide a skin-attachable transparent electrode that has excellent adhesion, which enables attachment to the skin without a need for a separate adhesive, does not cause skin irritation upon detachment, and allows for easy control of electrical characteristics, and a method for manufacturing the same.

Still another object of the present invention is to provide a wearable electronic device including the skin-attachable transparent electrode.

Yet another object of the present invention is to provide a wound healing pad including the skin-attachable transparent electrode.

Still yet another object of the present invention is to provide a wound healing method using the skin-attachable transparent electrode.

### Technical Solution

A skin-attachable transparent electrode according to the present invention includes an alcohol-soluble biocompatible elastomer matrix and a conductive material network embedded in the matrix, in which one surface of the biocompatible elastomer matrix is dissolved upon contact with alcohol and is conformally coated on skin.

In an implementation, one surface of the biocompatible elastomer matrix may be conformally coated along a shape of pores of the skin.

In an implementation, the conductive material may be one or a combination of two or more selected from the group consisting of a metal nanowire, a metal nanoparticle, a metal nanomesh, a carbon nanotube, a graphene-based compound, graphite, and a conductive polymer.

In an implementation, the conductive material may include a one-dimensional conductive material.

In an implementation, the conductive material may include a metal nanowire.

In an implementation, the biocompatible elastomer may be a thermoplastic polymer with a glass transition temperature of −10° C. or lower.

In an implementation, the biocompatible elastomer may include polyurethane.

In an implementation, the polyurethane may include a polyether-based diol structural unit.

In an implementation, the alcohol may be C1-3 alcohol.

In an implementation, the biocompatible elastomer may be water-insoluble.

In an implementation, the biocompatible elastomer may have a Young's modulus of 500 kPa or less.

In an implementation, an elongation at break of the biocompatible elastomer may be greater than an elongation at break of the biocompatible elastomer coated with alcohol and less than an elongation at break of the biocompatible elastomer coated with distilled water.

In an implementation, the skin-attachable transparent electrode may have a light transmittance of 65% or higher at 550 nm˜700 nm.

In an implementation, the skin-attachable transparent electrode may be used for wound healing by electrical stimulation.

In an implementation, the wound may be a chronic wound.

In addition, the present invention may provide a wearable electronic device including the skin-attachable transparent electrode.

In an implementation, the wearable electronic device may be a sensor, an electronic skin, a flexible display, or a stretchable display.

In an implementation, the wearable electronic device may include a function generator configured to detect a physiological signal and adjust a voltage, frequency, time, or waveform of electrical stimulation.

In addition, the present invention may provide a wound healing pad including the skin-attachable transparent electrode.

In an implementation, the wound healing pad may be substantially free of an adhesive.

In an implementation, the wound healing pad may be configured to heal a wound by electrical stimulation.

In an implementation, the wound healing pad may include two or more skin-attachable transparent electrodes positioned spaced apart from each other and a power supplying unit configured to electrically connect the two or more skin-attachable transparent electrodes.

In an implementation, the wound healing pad may further include a function generator electrically connected between the two or more skin-attachable transparent electrodes.

In the wound healing pad according to an implementation, the two or more skin-attachable transparent electrodes may be positioned spaced apart from each other with a wound interposed therebetween.

In addition, the present invention may provide a method for manufacturing a skin-attachable transparent electrode, the method including: forming a self-assembled monolayer on a substrate; forming a conductive material network on the self-assembled monolayer; and applying and drying an alcohol-soluble biocompatible elastomer solution on the substrate where the conductive material network is formed, thereby manufacturing a conductive material network embedded in an alcohol-soluble biocompatible elastomer matrix.

In an implementation, the forming a self-assembled monolayer may include coating a solution for forming a self-assembled monolayer on the substrate; and annealing the substrate coated with the solution for forming a self-assembled monolayer.

In an implementation, the solution for forming a self-assembled monolayer may be an alkoxysilane-based compound substituted with a fluorine group or a chlorosilane-based compound substituted with a fluorine group.

In an implementation, the method for manufacturing a skin-attachable transparent electrode may further include separating the conductive material network embedded in the alcohol-soluble biocompatible elastomer matrix from the substrate where the self-assembled monolayer is formed.

In addition, the present invention may provide a wound healing method including: treating a skin site to which a skin-attachable transparent electrode is to be attached with alcohol; attaching the skin-attachable transparent electrode to the skin site treated with alcohol; and applying electrical stimulation to the skin-attachable transparent electrode.

In the wound healing method according to an implementation, the skin-attachable transparent electrode may include two or more skin-attachable transparent electrodes positioned spaced apart from each other with a wound site interposed therebetween.

In the wound healing method according to an implementation, the electrical stimulation may be generated by a function generator.

### Advantageous Effects

The skin-attachable transparent electrode according to the present invention not only has remarkably excellent adhesion to the skin, biocompatibility, light transmittance, and conformal contact properties with the skin, but also can significantly improve stability because it is not dissolved by external environments such as sweat. Accordingly, it is possible to provide a wearable electronic device and a wound healing pad with superior performance.

### DESCRIPTION OF THE DRAWINGS

FIG. 1 is a schematic view of a skin-attachable transparent electrode according to an embodiment.

FIG. 2 is a schematic view of a wound healing pad according to an embodiment.

FIG. 3 is a view showing a method for manufacturing a skin-attachable transparent electrode according to an embodiment.

FIG. 4 is a view showing results of a skin irritation test of a skin-attachable transparent electrode according to an embodiment.

FIGS. 5 to 7 are views showing results of a cell culture test.

FIG. 8 shows stress-strain curves of PDMS, PVA, and PU.

FIG. 9 is a view showing results of a tensile test performed after spraying ethanol or deionized water on PDMS, PVA, and PU.

FIGS. 10 to 13 are views showing results of an adhesive force evaluation of a skin-attachable transparent electrode according to an embodiment.

FIG. 14 is a SEM image of an electrode according to Example 1.

FIG. 15 is a view showing transmittance of the electrode according to Example 1.

FIG. 16 is a view showing surface resistance according to Example 1.

FIG. 17 is a view showing changes in electrical characteristics caused by external environments.

FIGS. 18 and 19 are views showing evaluation results of conformal contact properties with the skin after manufacturing a skin-attachable transparent electrode according to an embodiment as a strain sensor.

FIG. 20 is a view showing an experimental method for impedance analysis, and FIG. 21 is a view showing results thereof.

FIG. 22 is a design schematic view of an ECG sensor that can be mounted on one arm.

FIG. 23 is a view showing measurement results of a Lead 1 ECG sensor using the electrode according to Example 1.

FIG. 24 is a view showing measurement results of an ECG sensor that can be mounted on one arm.

FIG. 25 is a view showing an experimental method for measuring EMG signals, and FIG. 26 is a view showing results thereof.

### MODE FOR INVENTION

The embodiments described in the present specification may be modified in many different forms, and the technology according to an implementation is not limited to the embodiments set forth below. In addition, the embodiments of an implementation are provided so that the present disclosure will be described in more detail to one skilled in the art.

In addition, the singular form used in the specification and claims appended thereto may be intended to include a plural form also, unless otherwise indicated in the context.

In addition, the numerical range used in the present specification includes all values within the range, including the lower limit and the upper limit, increments logically derived in a form and span of a defined range, all double limited values, and all possible combinations of the upper limit and the lower limit within the numerical range defined in different forms. Unless otherwise defined herein, values outside the numerical range that may arise due to experimental errors or rounded values are also included in the defined numerical range.

In addition, unless explicitly described to the contrary, the word “include or comprise”, and variations such as “includes or comprises” or “including or comprising”, will be understood to imply the inclusion of stated elements but not the exclusion of any other elements.

The present inventors have recognized that skin-attachable transparent electrode materials of the related art, even if they satisfy properties such as adhesiveness and transmittance, are unsuitable for application to wearable electronic devices, wound healing pads, and the like due to problems such as dissolution caused by sweat and insufficient conformal contact properties with the skin. As a result of repeated research to solve the problems, the present inventor discovered that a skin-attachable transparent electrode including a conductive material network embedded in an alcohol-soluble biocompatible elastomer matrix resolves the above-mentioned problems and has remarkably excellent adhesion to the skin, biocompatibility, and light transmittance, thereby completing the present invention.

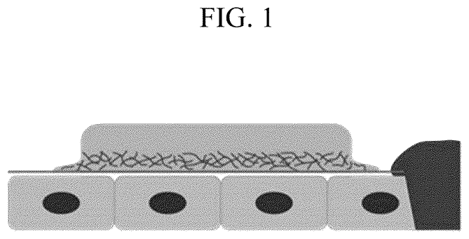

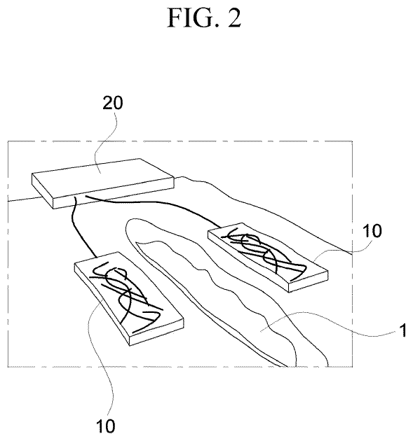

FIG. 1 is a schematic view of a skin-attachable transparent electrode according to an embodiment.

A skin-attachable transparent electrode according to an embodiment of the present disclosure will be described in detail below with reference to the accompanying drawings. The accompanying drawings are provided by way of example only to ensure that the technical spirit of the present invention can be sufficiently conveyed to technicians, and the present invention is not limited to the drawings presented below and may be embodied in many other forms.

Referring to FIG. 1, a skin-attachable transparent electrode according to an embodiment of the present invention includes an alcohol-soluble biocompatible elastomer matrix and a conductive material network embedded in the matrix, in which one surface of the biocompatible elastomer matrix is dissolved upon contact with alcohol and is conformally coated on skin.

The skin-attachable transparent electrode according to an embodiment includes the conductive material network embedded in the alcohol-soluble biocompatible elastomer matrix, so that one surface of the biocompatible elastomer matrix is dissolved upon contact with alcohol and can be conformally coated on the skin. In addition, the skin-attachable transparent electrode can have excellent adhesive force that enables attachment to the skin without a need for a separate adhesive, and therefore, does not cause problems of skin irritation caused by an adhesive and reduced electrical signal detectivity.

In an implementation, one surface of the biocompatible elastomer matrix may be conformally coated along a shape of pores of the skin. Accordingly, the skin-attachable transparent electrode can provide a wearable electronic device and a wound healing pad with further improved signal detectivity and superior performance.

In an implementation, the conductive material may be one or a combination of two or more selected from the group consisting of a metal nanowire, a metal nanoparticle, a metal nanomesh, a carbon nanotube, a graphene-based compound, graphite, and a conductive polymer.

Metal of the metal nanowire, metal nanoparticle or metal nanomesh may include, for example, silver (Ag), gold (Au), platinum (Pt), copper (Cu), aluminum (Al) or an alloy thereof, and specifically, may include silver.

The conductive polymer may include one or a mixture of two or more selected from the group consisting of poly(3,4-ethylenedioxythiophene):

poly (styrenesulfonate), polyethylenedioxythiophene, polyaniline, polypyrrole, polythiophene, polyp-phenylene, polyp-phenylenevinylene, polyacetylene, polydiacetylene, polythiophenevinylene, polyfullerene, and derivatives thereof, and preferably, poly(3,4-ethylenedioxythiophene):poly(styrenesulfonate) may be used, but no such limitation is intended.

In an implementation, the conductive material may include a one-dimensional conductive material, and preferably may include a metal nanowire. A diameter of the metal nanowire may be, but is not limited to, 10 nm to 500 nm, specifically 20 nm to 300 nm, and more specifically 20 nm to 100 nm, and an aspect ratio may be, but is not limited to, 60 to 3000, specifically 100 to 1500, and more specifically 300 to 1500.

In an implementation, the biocompatible elastomer may be a thermoplastic polymer with a glass transition temperature of−10° C. or lower, and specifically, the glass transition temperature may be −20° C. or lower or −30° C. or lower.

The biocompatible elastomer may include polyurethane, polyoxyethylene-polybutylene terephthalate copolymer, styrene-butadiene copolymer (styrene-butadiene rubber, SBR), styrene-ethylene-butylene-styrene copolymer (styrene-ethylene-butylene-styrene, SEBS), styrene-ethylene-propylene-styrene copolymer (styrene-ethylene-propylene-styrene, SEPS), styrene-butadiene-styrene copolymer (styrene-butadiene-styrene, SBS), styrene-isoprene-styrene copolymer (styrene-isoprene-styrene, SIS), styrene-isobutylene-styrene copolymer (styrene-isobutylene-styrene, SIBS) or combination thereof, but is not limited thereto as long as it is an alcohol-soluble elastomer. Preferably, the biocompatible elastomer may include polyurethane.

In an implementation, the polyurethane may include a polyether-based diol structural unit, and specifically, may include a polytetramethylene ether diol structural unit. This makes it possible to achieve the object of the present invention more preferably by having the properties of being soluble in alcohol but not in an aqueous solution. The biocompatible elastomer may also include structural units derived from alicyclic isocyanate-based monomers, such as, but not limited to, isophorone diisocyanate or 4,4-dicyclohexylmethane diisocyanate.

In an implementation, the alcohol capable of dissolving the biocompatible elastomer may be specifically C1-3 alcohol, and more specifically ethanol.

In an implementation, the biocompatible elastomer may be water-insoluble, so that, after a skin-attachable transparent electrode including the same is dissolved in alcohol and effectively attached to the skin, it is not dissolved by water-soluble substances such as sweat or rain, thereby enhancing the durability of a product to which it is applied.

In an implementation, the biocompatible elastomer may have a Young's modulus of 500 kPa or less, 400 kPa or less, 300 kPa or less, or 250 kPa or less, and the lower limit may be, for example, 100 kPa or 150 kPa. In an implementation, an elongation at break of the biocompatible elastomer may be greater than an elongation at break of the biocompatible elastomer coated with alcohol and less than an elongation at break of the biocompatible elastomer coated with distilled water.

In this case, the decrease in the elongation at break of the biocompatible elastomer when alcohol is applied is interpreted as a result of the biocompatible elastomer being dissolved in alcohol, which weakens bonds among molecules of the biocompatible elastomer, whereas the increase in the elongation at break of the biocompatible elastomer when distilled water is applied is interpreted as a result of the biocompatible elastomer not being dissolved in distilled water but rather forming firm bonds among molecules of the biocompatible elastomer.

In an implementation, a thickness of the matrix may be 100 μm to 200 μm, specifically 150 μm to 200 μm, and more specifically 160 μm to 180 μm. Within the above range, the physical properties of the skin-attachable transparent electrode, such as light transmittance and adhesiveness, can be implemented.

In an implementation, a thickness of the conductive material network may be 10 nm to 500 nm, specifically 10 nm to 250 nm, and more specifically 50 nm to 150 nm.

In an implementation, a ratio of the thickness of the conductive material network to the thickness of the matrix may be 1:1000 to 2000, 1:1500 to 2000, or 1:1600 to 1800. Within this range, the light transmittance, adhesiveness, electrical conductivity, and the like of the skin-attachable transparent electrode as well as stability against external environments can be further improved.

In an implementation, the conductive material network may be embedded in the matrix so as to be in contact with one surface of the matrix that is dissolved upon contact with alcohol and is conformally coated on the skin, which positions the conductive material network in the direction of the surface in contact with the skin, thereby further improving signal detectivity.

When the conductive material network is embedded inside the matrix so as to be in contact with one surface of the matrix, the conductive material network may be completely or partially embedded in the matrix, and preferably may be partially embedded. More preferably, the conductive material network may be exposed on one surface of the matrix and the conductive material network may not be exposed on the other surface of the matrix. This allows the surface portion where the conductive material network is exposed to adhere to the skin, enhancing electrical properties and thereby enabling more effective detection of physiological signals or improved healing effects through electrical stimulation.

In an implementation, the skin-attachable transparent electrode may have a light transmittance of 65% or higher or 70% or higher at wavelengths ranging from 550 nm to 700 nm, and the upper limit may be, for example, 85% or 90%.

In an implementation, the skin-attachable transparent electrode may be used for wound healing by electrical stimulation, and the wound refers to a condition where the skin and subcutaneous tissue are damaged due to external stimuli. The wound may include both a minor wound that can heal naturally and a chronic wound, and specifically, the wound may be a chronic wound.

The present invention provides a wearable electronic device including the skin-attachable transparent electrode as described above.

In an implementation, the skin-attachable transparent electrode may be applied to various sensors, including strain sensors, temperature sensors, pressure sensors, optical sensors, vibration sensors, and biosensors, and various wearable electronic devices such as electronic skins, flexible displays, and stretchable displays, and in particular, may be applied to body-attachable wearable electronic devices designed for a curved surface of the skin, but this does not restrict its application to clothing-type or accessory-type wearable electronic devices.

In an implementation, the sensor may include a function generator that detects a physiological signal and adjusts a voltage, frequency, time or waveform of electrical stimulation, in which the physiological signal may include, but is not limited to, an electrocardiogram, a electromyogram, and body movement. The stimulation may have a form such as a sine wave, a pulse, or a square wave.

In addition, the present invention provides a wound healing pad including the skin-attachable transparent electrode as described above. FIG. 2 is a schematic view of the wound healing pad according to an embodiment, and the wound healing pad according to the present disclosure will be described in detail with reference to FIG. 2.

A wound healing pad according to an implementation may include two or more skin-attachable transparent electrodes 10 positioned spaced apart from each other and a power supplying unit 20 that electrically connects the two or more skin-attachable transparent electrodes. Specifically, the two or more transparent electrodes may be positioned spaced apart from each other with a wound 1 interposed therebetween.

In an implementation, the wound healing pad may further include a function generator electrically connected between the two or more skin-attachable transparent electrodes, in which the function generator serves to adjust a voltage, frequency, time, or waveform of electrical stimulation. By adjusting the characteristics of the electrical stimulation, the degree of treatment can be determined depending on a location, size, depth, and shape of the wound, the patient's recovery status, and the like.

In an implementation, the wound healing pad may be substantially free of an adhesive by including the skin-attachable transparent electrode. More specifically, the inclusion of the alcohol-soluble biocompatible elastomer significantly improves the adhesive force to the skin, which enables effective attachment to the skin without a need for a separate adhesive and does not cause the problems of skin irritation and reduced electric signal detectivity caused by an adhesive.

Note that the description “substantially free of an adhesive” means that an adhesive is not included in an amount that would substantially affect the operation of the skin-attachable transparent electrode or wound healing pad, and does not exclude the inclusion of trace amounts that may be present as impurities or for other known supplementary effects. Specifically, the adhesive may be included in an amount of 1 wt % or less, 0.1 wt % or less, 0.01 wt % or less, or 0.001 wt % or less, with a lower limit being 0 wt % or more, with respect to a total weight of the wound healing pad.

In an implementation, the wound healing pad may heal a wound by electrical stimulation, and the wound may specifically be a chronic wound.

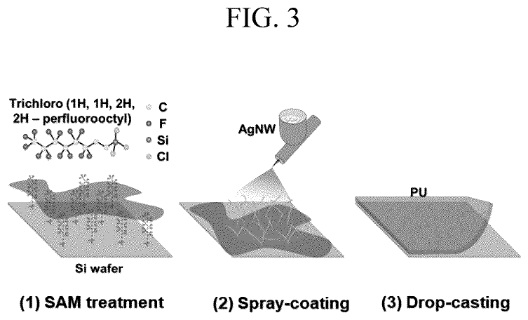

FIG. 3 is a view showing a method for manufacturing a skin-attachable transparent electrode according to an embodiment. Below, the method for manufacturing a skin-attachable transparent electrode will be described in detail with reference to FIG. 3.

A method for manufacturing a skin-attachable transparent electrode according to an implementation of the present invention includes a first step of forming a self-assembled monolayer on a substrate; a second step of forming a conductive material network on the self-assembled monolayer; and a third step of applying and drying an alcohol-soluble biocompatible elastomer solution on the substrate where the conductive material network is formed, thereby manufacturing a conductive material network embedded in an alcohol-soluble biocompatible elastomer matrix.

The first step is a step of forming a self-assembled monolayer on a substrate, and the substrate may include a transparent material that can transmit light, and may include, for example, a silicon substrate, a glass substrate, or a polymer substrate, but is not limited thereto.

The silicon substrate may include a single silicon substrate or a p-Si substrate, and the glass substrate may be made of one or a combination of alkaline silicate glass, non-alkali glass, or quartz glass, but is not limited thereto and may be made of various other materials.

The polymer substrate may be made of one or a combination of polyethylene terephthalate (PET), polyethylene naphthalate (PEN), polyimide (PI), and polyurethane, but is not limited thereto and may be made of various other materials. However, the polymer substrate is not necessarily limited thereto as long as it has transparency and flexibility sufficient to be used in transparent flexible displays.

In an implementation, the first step may include steps of coating a solution for forming a self-assembled monolayer on the substrate; and annealing the substrate coated with the solution for forming a self-assembled monolayer.

Specifically, the solution for forming a self-assembled monolayer may be a solution including a silane-based compound. The silane-based compound may be expressed by Si(R¹)₄₋ₙR²ₙ, where R¹ is hydroxy, C₁-C₄ alkoxy or halogen, R² is C₁-C₂₀ alkyl, C₁-C₂₀ carboxyalkyl, C₁-C₂₀ aminoalkyl, C₁-C₂₀ perfluoroalkyl, C₁-C₂₀ fluoroalkyl or C₂-C₂₀ acryloxyalkyl, and n is an integer of 1 to 3. Specifically, the silane-based compound may be an alkoxysilane-based compound or a chlorosilane-based compound, and more specifically, an alkoxysilane-based compound substituted with a fluorine group or a chlorosilane-based compound substituted with a fluorine group so that the surface can be treated to be hydrophobic.

Specifically, the annealing may be performed at 100° C. to 180° C. or 100° C. to 150° C.

The second step is a step of forming a conductive material network on the self-assembled monolayer, and the above description can be applied to the conductive material. Specifically, the second step may include a step of coating a conductive material solution onto the self-assembled monolayer. The coating method may use, for example, spin coating, spray coating, inkjet coating, slit coating, deep coating, or the like, but is not particularly limited.

The third step is a step of applying and drying an alcohol-soluble biocompatible elastomer solution on a substrate where the conductive material network is formed, thereby manufacturing an alcohol-soluble biocompatible elastomer matrix in which the conductive material network is embedded. The above description can be applied to the alcohol-soluble biocompatible elastomer. The applying may be performed by various methods for forming a thin film using a solution process, and preferably may be performed by at least one method selected from spin coating, drop casting, dip coating, spray coating, flow casting, screen printing, inkjet printing, and micro-contact printing, and more preferably may be performed by drop casting.

In an implementation, the method for manufacturing a skin-attachable transparent electrode may further include a fourth step of separating the conductive material network embedded in the alcohol-soluble biocompatible elastomer matrix from the substrate where the self-assembled monolayer is formed.

A wound healing method according to an implementation of the present invention includes the steps of: treating a skin site to which a skin-attachable transparent electrode is to be attached with alcohol; attaching the skin-attachable transparent electrode described above to the skin site treated with alcohol; and applying electrical stimulation to the skin-attachable transparent electrode.

In an implementation, the skin-attachable transparent electrode may include two or more skin-attachable transparent electrodes positioned spaced apart from each other with a wound site therebetween.

In an implementation, the electrical stimulation may be generated by a function generator.

The wound healing method according to an implementation is such that one surface of the biocompatible elastomer matrix included in the skin-attachable transparent electrode described above is dissolved upon contact with alcohol and is conformally attached to the skin, improving signal detectivity and thereby enabling more effective wound healing. In addition, by using the transparent electrode with excellent adhesive force that enables attachment to the skin without a need for a separate adhesive, a wound can be healed without the problems of skin irritation and reduced electrical signal detectivity caused by an adhesive.

### DETAILED DESCRIPTION OF THE EMBODIMENTS

Hereinafter, examples and experimental examples will be exemplified specifically in detail in the following. However, the examples and experimental examples described below are only illustrative of part, and the technology described in the present specification is not construed as being limited thereto.

### <Example 1> Preparation of Skin-Attachable Transparent Electrode

First, self-assembled monolayers (SAMs) formed on a Si wafer substrate were formed by evaporating a solution of trichloro(1H,1H,2H,2H-perfluorooctyl) silane (Sigma Aldrich) in a vacuum chamber where the Si wafer substrate was placed and performing annealing at 135° C. for 1 hour.

A dispersion was prepared by dispersing 0.63 wt % silver nanowires (Novarials Corporation) with an average diameter of 30 nm and a length of 30 μm in ethanol, the dispersion was evaporated, and then 0.3 ml was spray-coated on the substrate (2.5 cm×2.5 cm) on which the SAMs were formed to form a silver nanowire network on the SAMs. Specifically, the distance between the nozzle and the substrate was 15 cm and the spray coating was performed at a speed of 0.3 ml min⁻¹.

Thereafter, 10 wt % of polyether-based hydrophilic PU (Hydromed D4 available from AdvanSource Biomaterials Corporation) was dissolved in a solvent with a weight ratio of ethanol to distilled water of 19:1 to prepare a PU solution. The PU solution was drop-cast onto the substrate with the silver nanowire network formed thereon and then dried at room temperature for 4 hours to prepare a PU matrix with the silver nanowire network embedded therein.

Finally, the prepared PU matrix with the silver nanowire network embedded therein was separated from the substrate on which the SAMs were formed at room temperature to prepare a skin-attachable transparent electrode (indicated as Ag/PU or TSE in the drawing).

### <Experimental Example 1> Biocompatibility Evaluation

Biocompatibility evaluation was performed through a skin irritation test and a cell culture test.

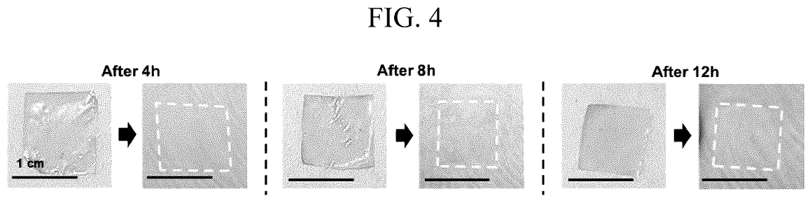

For the skin irritation test, the electrode prepared in Example 1 was attached to the skin, and then removed after 4 hours, 8 hours, and 12 hours to measure the degree of skin irritation, and the results are shown in FIG. 4. Referring to FIG. 4, no erythema, residue, or pain occurred at the site (indicated by the white dotted line) where the electrode prepared in Example 1 was removed, confirming that the skin-attachable transparent electrode according to an embodiment has low skin irritation.

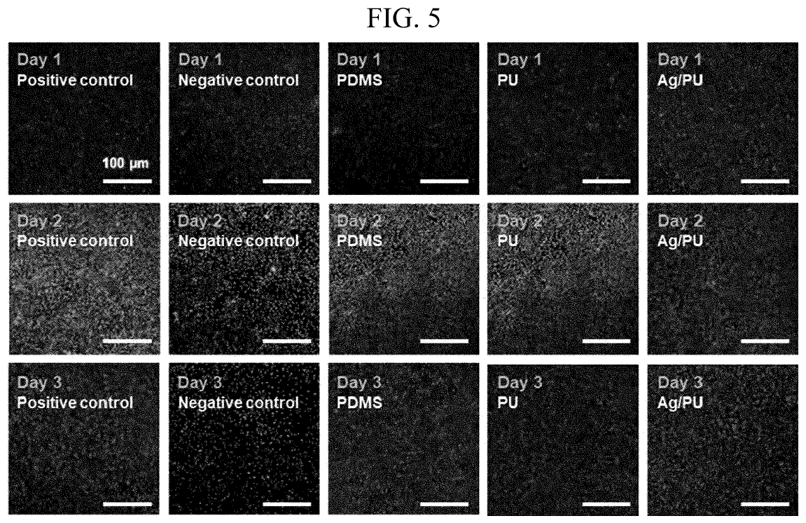

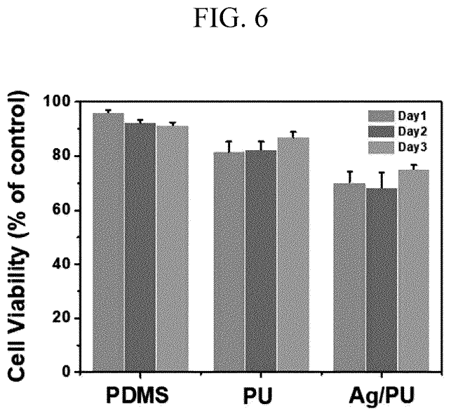

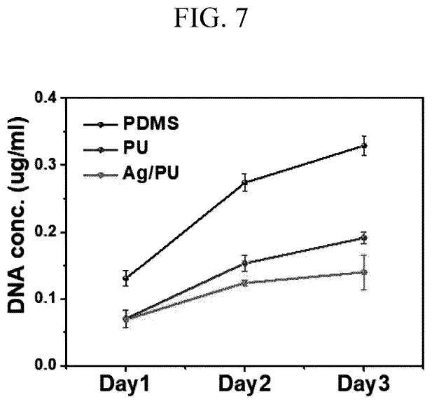

For the cell culture test, the electrode (or Ag/PU) prepared in Example 1, polyurethane (PU; Hydromed D4 available from AdvanSource Biomaterials Corporation), and poly(dimethylsiloxane) (PDMS; Sylgard 184 elastomer kit, Dow Corning) were added, respectively, and mouse fibroblasts (L929) were cultured. The cell culture medium was 2 ml of high glucose Dulbecco's modified Eagle's medium (DMEM) containing 10% fetal bovine serum (FBS) and 1% antibiotics, and was observed, under the culture conditions of 37° C., 5% CO₂, and 95% humidity, for 3 days through daily live/dead cell staining (n=1), cell viability test by CCK8 assay (n=2), and proliferation test by DNA content assay (PicoGreen, n=3). Additionally, the positive control was cultured in the cell culture medium alone, and the negative control was cultured in the cell culture medium supplemented with antimycin A. The results are shown in FIGS. 5 to 7, respectively. Referring to FIG. 5, the fluorescent live/dead staining image shows the regular cell morphology similar to the positive control in all cases after 3 days, indicating that the material has high cell compatibility and is non-toxic to cells. Referring to FIG. 6, for PDMS and PU, the cell viability was over 80% from day 1, whereas for Ag/PU, the cell viability was lower at 70% on day 1 but continuously increased over time. In this case, the cell viability refers to a ratio (NA/NB) of the number of cells grown on each sample surface (NA) to the number of cells grown in the positive control (NB). Referring to FIG. 7, all cases showed an increasing trend in DNA concentration over 3 days, confirming the biocompatibility in terms of cell proliferation.

### <Experimental Example 2> Mechanical Properties Evaluation

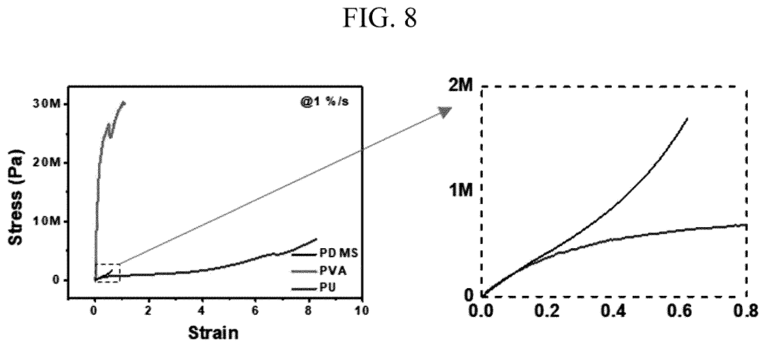

To evaluate the mechanical properties, PDMS, PVA, and PU were prepared in a size of 2 cm×0.5 cm and subjected to tensile tests. The tensile tests were performed using a Universal Testing Machine (UTM, WL2100C, WithLab) with a 1 kgf load cell, and the results are shown in FIG. 8. FIG. 8 shows the stress-strain curves of each polymer material. Referring to FIG. 8, the Young's modulus of PU was less than 225 kPa, which was much lower than those of PDMS and PVA.

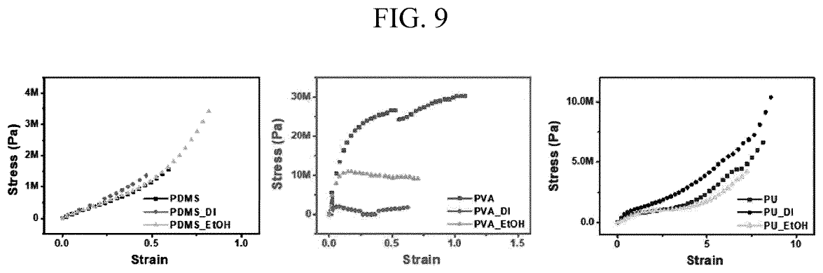

In addition, to confirm the effect of solvent on each polymer material, a tensile test was performed after spraying ethanol or deionized water, and the results are shown in FIG. 9. Referring to FIG. 9, PDMS did not exhibit a reduced elongation at break due to deionized water or ethanol, indicating that deionized water and ethanol cannot alter the main polymer chains of PDMS. In contrast, PVA was dissolved in both deionized water and ethanol, so the elongation at break was reduced from 120% to 70% and the tensile strength was lowered. PU showed a more rapid reduction in elongation at break from 800% to 700% only when treated with ethanol, whereas treatment with deionized water did not break the covalent bonds in the polymer. With this, it can be seen that the PU material is soluble in alcohol but not in deionized water, so it can be used stably even in external environments such as sweat, making it suitable for use as a skin-attachable electrode.

### <Experimental Example 3> Adhesive Force Evaluation

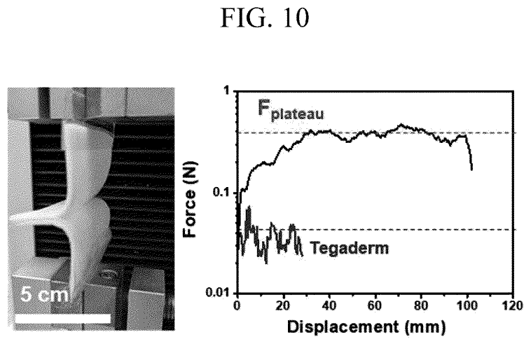

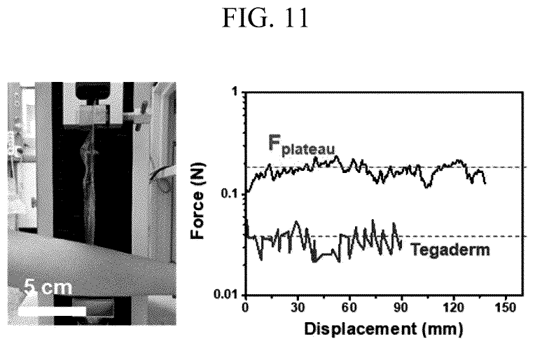

To evaluate the adhesive force of the electrode, a 180° peel test (ASTM F2256) on artificial skin and a 90° peel test (ASTM D6862) on human forearm were performed using a UTM (WL2100C, WithLab) with a 1 kgf load cell. As the artificial skin, Micropig Franz Cell Membrane (FCM) available from APURES was used, and the electrode (50 mm×50 mm) prepared in Example 1 was placed between two FCMs, sprayed with ethanol, and attached by applying gentle pressure. Thereafter, the electrode prepared in Example 1 was peeled at a peeling speed of 50 mm min−1, and the results are shown in FIG. 10. Additionally, the 90° peel test was performed on a human forearm using the electrode prepared in Example 1 with a size of 50 mm×150 mm, and the results are shown in FIG. 11. Referring to FIGS. 10 and 11, the electrode prepared in Example 1 showed the interfacial toughness of 0.08 N cm⁻¹ on the artificial skin and 0.05 N cm⁻¹ on the human forearm. In addition, the same test was performed using commercially available Tegaderm film. The measured interfacial toughness values for Tegaderm were 0.008 N cm⁻¹ and 0.007 N cm⁻¹ cm on the artificial skin and the human forearm, respectively. According to the reported interfacial toughness values for elastomers such as EcoFlex (00-30, Smooth-On, <0.04 N cm⁻¹) and PDMS (Sylgard 184, Dow Corning, <0.04 N cm⁻¹), the skin-attachable electrode according to an embodiment demonstrates excellent adhesion properties on both the artificial skin and the human skin.

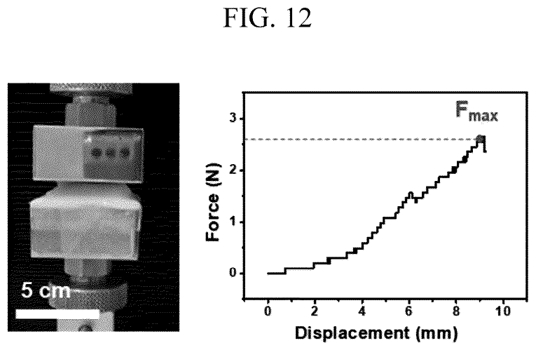

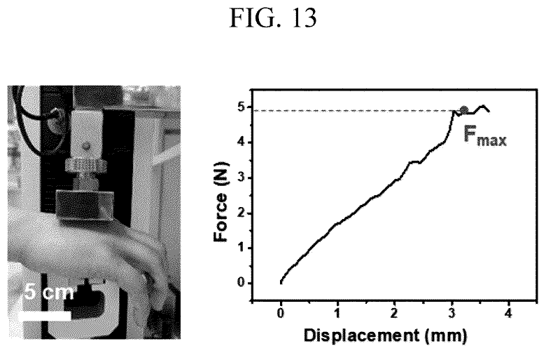

Additionally, the tensile test was performed on the FCM with a contact area of 30 mm×30 mm and the back of the hand with a contact area of 50 mm×50 mm, and the results are shown in FIGS. 12 and 13, respectively. Referring to FIGS. 12 and 13, the electrode prepared in Example 1 exhibited the tensile strength of 0.3 N cm⁻² and 0.2 N cm⁻² on the artificial skin and the human skin, respectively.

### <Experimental Example 4> Evaluation of Transmittance and Electrical Characteristics

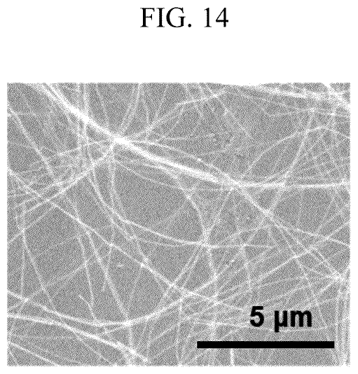

The transmittance was measured using UV-visible spectroscopy. The sheet resistance was measured using a four-point probe. Additionally, the electrode after peeling from the skin peeling was analyzed using field-emission scanning electron microscopy (FE-SEM, IT500, JEOL Ltd). In addition, to evaluate chemical stability, changes in electrical characteristics due to the external environment were observed, and specifically, the skin-attachable electrode according to Example 1 was exposed to deionized water, pH 4.01 buffer solution (Reagent Duksan), pH 6.86 buffer solution (Reagent Duksan), and ambient air (ambient condition) to measure the rate of change in resistance value. The resistance was measured using a digital multimeter (Fluke) every 4 hours for up to 12 hours. The results are shown in FIGS. 14 to 17.

FIG. 14 is an SEM image of an electrode according to Example 1. Referring to FIG. 14, it can be confirmed that the silver nanowires stably penetrate and are embedded in the polyurethane phase after being separated from the substrate.

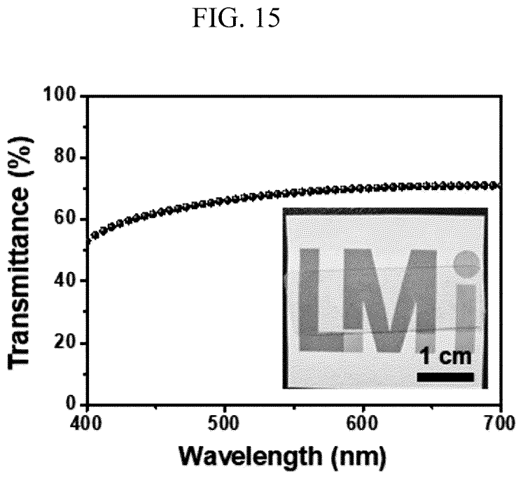

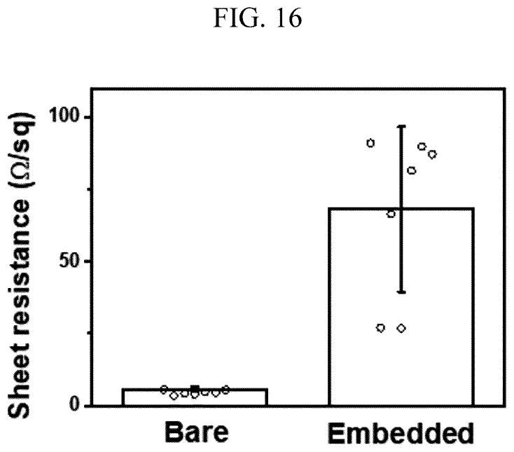

Note that the amount of silver nanowires used is adjusted by applying a spray coating method as a coating method, which enables the transmittance and surface resistance to be adjusted. FIGS. 15 and 16 show the transmittance and surface resistance of the electrode according to Example 1, respectively. Referring to FIGS. 15 and 16, it can be confirmed that the sample with surface resistance of 30 ohm sq-1 shows about 70% light transmittance at the wavelength of 550 nm, and the surface resistance of the electrode with silver nanowires embedded in PU (indicated as Embedded in FIG. 16) increases to 70 ohm sq-1 compared to the electrode with silver nanowires coated on the glass substrate (indicated as Bare in FIG. 16). This is because mechanical stimulation was applied to the PU when the PU electrode with silver nanowires embedded therein was separated from the substrate.

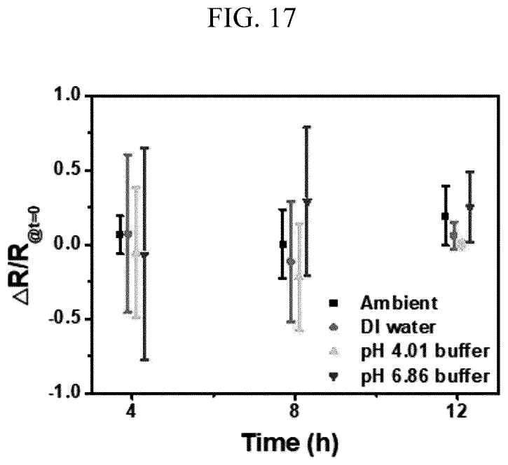

FIG. 17 is a view showing changes in electrical characteristics caused by external environments. Referring to FIG. 17, the relative resistance value (R/Rt=0) changed less than 50%, and preferably less than 30%, after 12 hours of exposure to deionized water, pH 4.01 buffer solution, pH 6.86 buffer solution, and ambient air, respectively.

### <Experimental Example 5> Application to Strain Sensor

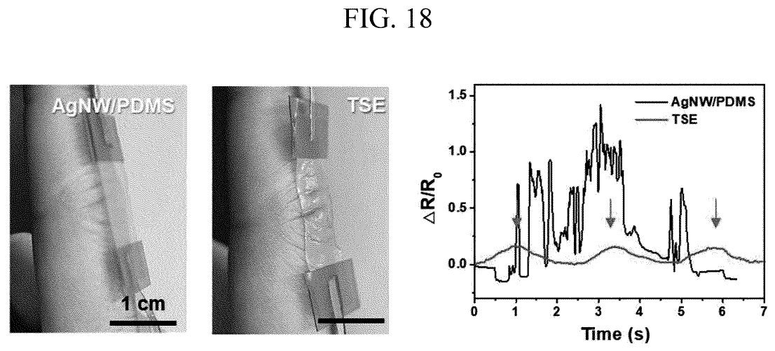

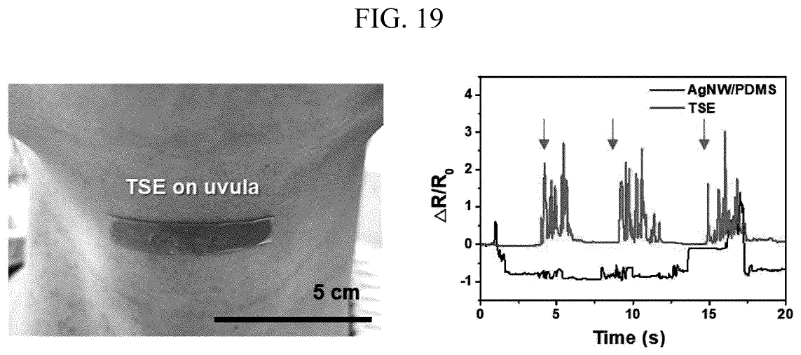

The skin-attachable transparent electrode (3 cm×0.5 cm) according to Example 1 was fabricated as a strain sensor for motion detection and its conformal contact properties with the skin were confirmed. An AgNW/PDMS electrode was used as a reference electrode. The AgNW/PDMS electrode (3 cm×0.5 cm) was prepared by spin-coating a dispersion, which was prepared by mixing a polydimethylsiloxane main agent and a polydimethylsiloxane curing agent in a weight ratio of 10:1, onto a glass substrate with AgNWs spray-coated thereon at 300 rpm for 15 seconds, followed by curing at 90° C. for 1 hour. The changes in resistance values were measured at the fingers and neck using a digital multimeter (DMM 6500, Keithley), and the results are shown in FIGS. 18 and 19. Referring to FIG. 18, it can be confirmed that the AgNW/PDMS electrode creates a gap and does not adhere to the wrinkles as the finger is bent, no change in resistance is observed at the finger bending cycles (indicated by the arrows in FIG. 18). In contrast, the electrode according to Example 1 can effectively transmit motion information to the strain sensor by achieving conformal contact with the generated wrinkles through the redissolution of the PU matrix on the surface by ethanol. Additionally, changes in resistance were clearly observed at the finger flexion cycles. FIG. 19 shows a case where the electrode was mounted on the uvula to detect the movement of drinking water, and likewise, the change in resistance was clearly observed when drinking water (indicated by the arrows in FIG. 19).

### <Experimental Example 6> Evaluation of Electrophysiological Characteristics

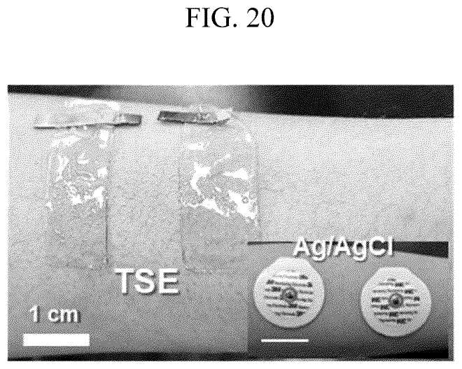

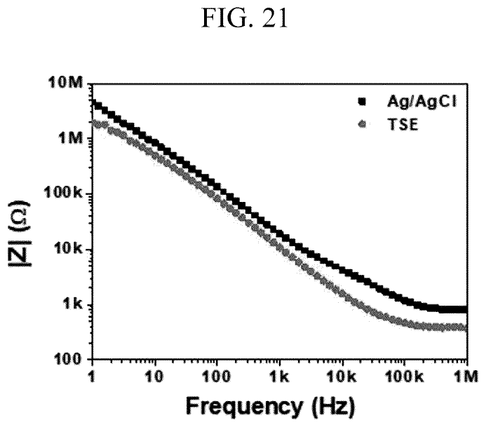

Impedance analysis, and ECG and EMG signal monitoring were performed to evaluate electrophysiological characteristics. The impedance analysis was performed by first preparing the electrodes (2 cm×2 cm) according to Example 1 and commercial Ag/AgCl gel electrodes (1.5 cm×2 cm) (2223H, 3M), attaching both the electrodes to the arm at 1 cm intervals as shown in FIG. 20, and connecting the electrodes to an electrochemical impedance spectrometer (SP-300, BioLogic) with a frequency ranging from 1 Hz to 1 MHz at 100 mV. The results of the impedance analysis are shown in FIG. 21. Referring to FIG. 21, it can be seen that the electrode according to Example 1 exhibits lower impedance than the Ag/AgCl electrode. With this, the skin-attachable transparent electrode according to an embodiment can improve the interfacial impedance characteristics.

Next, the ECG signal measurement was performed by connecting a data acquisition board (heart rate monitor sensor SKU: SEN 0213 from DFRobot) to Arduino UNO and using a commercial Ag/AgCl electrode as a reference electrode. The measurement was performed in two methods.

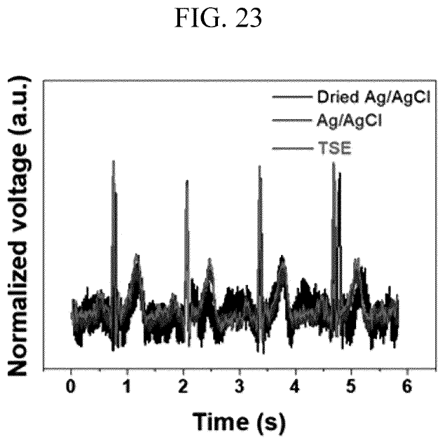

The first method was a conventional Lead 1 ECG measurement, which was performed by attaching the electrode according to Example 1, the Ag/AgCl electrode, and the dry Ag/AgCl electrode to the right arm, left arm, and left leg, and the results are shown in FIG. 23. The dry Ag/AgCl electrode was dried for 2 hours under ambient air conditions before attachment, and the signal-to-noise ratio was low because the hydrogel portion of the electrode was dried. In contrast, the electrode according to Example 1 (indicated as TSE in FIG. 23) showed a high signal-to-noise ratio similar to that of the Ag/AgCl electrode that did not undergo a drying process.

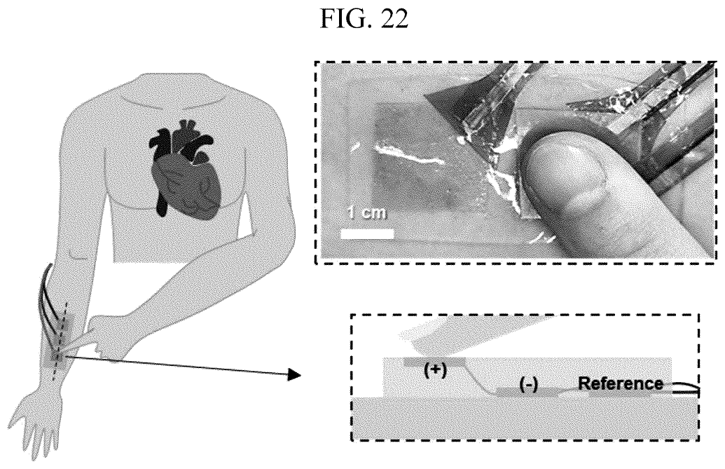

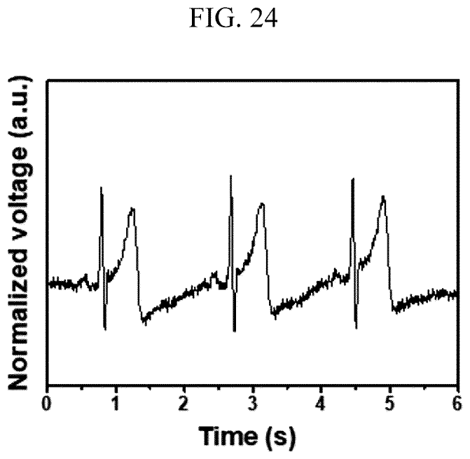

The second method was carried out by designing an ECG sensor that can be mounted on one arm as shown in FIG. 22. Specifically, two electrodes according to Example 1 and a reference electrode were attached to the right arm, and one of the electrodes according to Example 1 was touched with a finger of the left hand to detect an ECG signal, and the results are shown in FIG. 24. Referring to FIG. 24, the signal measured by the ECG sensor mounted on one arm had the form of a PQRSTU ECG signal that is typically obtained, but exhibited a much deeper TU valley than that of the conventional Lead 1 ECG measurement.

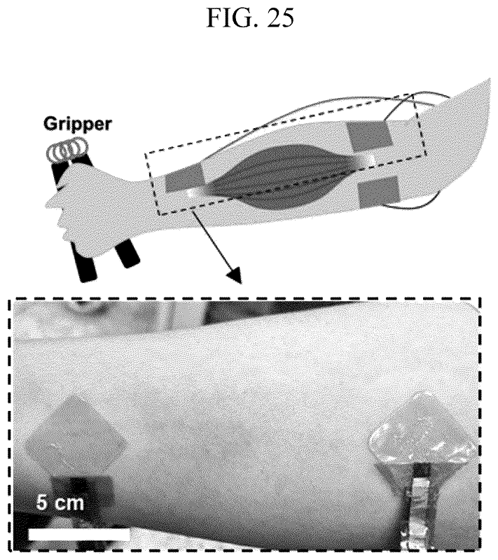

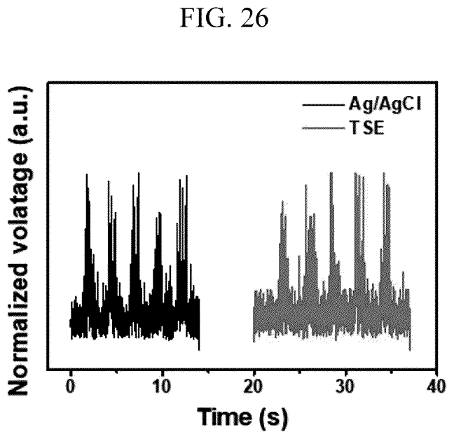

Finally, the EMG signal measurement was performed by connecting a data acquisition board (SZH-HWS010) to Arduino UNO and using a commercial Ag/AgCl electrode as a reference electrode, and two electrodes according to Example 1 were attached to the forearm and the reference electrode was attached to the upper arm, as shown in FIG. 25. The results of EMG signal measurement are shown in FIG. 26. Referring to FIG. 26, it can be seen that the electrode according to Example 1 can measure EMG signals with performance similar to that of the Ag/AgCl electrode.

With this, it can be seen that the skin-attachable transparent electrode according to an embodiment can excellently measure electrophysiological signals and does not have the problem associated with commercial Ag/AgCl electrodes, such as performance degradation due to drying. In addition, the skin-attachable transparent electrode according to an embodiment can be attached to the skin without the use of an adhesive that causes skin irritation and deterioration of electrical characteristics, has the advantages of excellent biocompatibility, light transmittance, and skin compliance, and can operate stably even in a sweaty state.

Hereinabove, although the present disclosure has been described by specific matters and limiting embodiments in the present specification, they have been provided only for assisting in the more general understanding of the present disclosure. Therefore, the present disclosure is not limited to the embodiments, and various modifications and changes can be made by one skilled in the art to which the present disclosure pertains from this description.

Therefore, the spirit described in the present specification should not be limited to the aforementioned embodiments, and the following claims as well as all modified equally or equivalently to the claims are intended to fall within the scope and spirit described in the present specification.

### REFERENCE SIGNS LIST

10: skin-attachable transparent electrode
20: power supplying unit
1: wound

1. A skin-attachable transparent electrode comprising:
an alcohol-soluble biocompatible elastomer matrix; and
a conductive material network embedded in the matrix,
wherein one surface of the biocompatible elastomer matrix is dissolved upon contact with alcohol and is conformally coated on skin.

2. The skin-attachable transparent electrode of claim 1, wherein:
one surface of the biocompatible elastomer matrix is conformally coated along a shape of pores of the skin.

3. The skin-attachable transparent electrode of claim 1, wherein:
a conductive material of the conductive material network is one or a combination of two or more selected from the group consisting of a metal nanowire, a metal nanoparticle, a metal nanomesh, a carbon nanotube, a graphene-based compound, graphite, and a conductive polymer.

4. The skin-attachable transparent electrode of claim 1, wherein:
the conductive material comprises a one-dimensional conductive material.

5. The skin-attachable transparent electrode of claim 4, wherein:
the conductive material comprises a metal nanowire.

6. The skin-attachable transparent electrode of claim 1, wherein:
a biocompatible elastomer of the biocompatible elastomer matrix is a thermoplastic polymer with a glass transition temperature of −10° C. or lower.

7. The skin-attachable transparent electrode of claim 1, wherein:
the biocompatible elastomer comprises polyurethane.

8. The skin-attachable transparent electrode of claim 7, wherein:
the polyurethane comprises a polyether-based diol structural unit.

9. The skin-attachable transparent electrode of claim 1, wherein:
the alcohol is C1-3 alcohol.

10. The skin-attachable transparent electrode of claim 1, wherein:
the biocompatible elastomer is water-insoluble.

11. The skin-attachable transparent electrode of claim 1, wherein:
the biocompatible elastomer has a Young's modulus of 500 kPa or less.

12. The skin-attachable transparent electrode of claim 1, wherein:
an elongation at break of the biocompatible elastomer is greater than an elongation at break of the biocompatible elastomer coated with alcohol and less than an elongation at break of the biocompatible elastomer coated with distilled water.

13. The skin-attachable transparent electrode of claim 1, wherein:
the skin-attachable transparent electrode has a light transmittance of 65% or higher at wavelengths ranging from 550 nm to 700 nm.

14. The skin-attachable transparent electrode of claim 1, wherein:
the skin-attachable transparent electrode is used for wound healing by electrical stimulation.

15. The skin-attachable transparent electrode of claim 14, wherein:
the wound is a chronic wound.

16-24. (canceled)

25. A method for manufacturing a skin-attachable transparent electrode, the method comprising:
forming a self-assembled monolayer on a substrate;
forming a conductive material network on the self-assembled monolayer; and
applying and drying an alcohol-soluble biocompatible elastomer solution on the substrate where the conductive material network is formed, thereby manufacturing a conductive material network embedded in an alcohol-soluble biocompatible elastomer matrix.

26. The method of claim 25, wherein:
the forming a self-assembled monolayer comprises coating a solution for forming a self-assembled monolayer on the substrate; and annealing the substrate coated with the solution for forming a self-assembled monolayer.

27. The method of claim 26, wherein:
the solution for forming a self-assembled monolayer is an alkoxysilane-based compound substituted with a fluorine group or a chlorosilane-based compound substituted with a fluorine group.

28. The method of claim 25, further comprising
separating the conductive material network embedded in the alcohol-soluble biocompatible elastomer matrix from the substrate where the self-assembled monolayer is formed.

29-31. (canceled)
